(19) United States
(12) Patent Application Publication
(10) Pub. No.: US 2018/0344150 A1
(43) Pub. Date: Dec. 6, 2018

(54) CONTROL APPARATUS, TOMOGRAPHIC IMAGE ACQUIRING SYSTEM, CONTROL METHOD, AND MEDIUM

(71) Applicant: CANON KABUSHIKI KAISHA, Tokyo (JP)

(72) Inventors: Tomasz Bajraszewski, Glogowo (PL); Hiroshi Hara, Machida-shi (JP); Hiroki Uchida, Tokyo (JP)

(21) Appl. No.: 15/989,496

(22) Filed: May 25, 2018

(30) Foreign Application Priority Data
Jun. 1, 2017 (JP) 2017-108992

(51) Int. Cl. A61B 3/10; A61B 3/12; A61B 3/14; G01B 9/02

(52) U.S. Cl. CPC A61B 3/102; A61B 3/1025; G01B 9/02091; A61B 3/14; A61B 3/1225

(57) ABSTRACT

Provided is a technology for acquiring a tomographic image under an appropriate focusing condition by an OCT apparatus. A control apparatus configured to control a tomographic image acquiring unit configured to acquire a tomographic image includes: a setting unit configured to set a focusing area on an object to be inspected; a condition acquiring unit configured to acquire a first focusing condition for a front image acquiring unit with respect to the focusing area, which is set in a partial region of a front image of the object to be inspected acquired by the front image acquiring unit, and is narrower than an image acquiring area of the front image; and a control unit configured to cause the tomographic image acquiring unit to acquire the tomographic image under a second focusing condition in accordance with the acquired first focusing condition.

### BACKGROUND OF THE INVENTION

### Field of the Invention

The present invention relates to a control apparatus configured to control an optical tomographic image acquiring apparatus to be used for ophthalmic diagnosis and treatment or other such purpose, a tomographic image acquiring system, a control method for the optical tomographic image acquiring apparatus, and a medium for storing a program for executing the control method.

### Description of the Related Art

Currently, various types of equipment are used as ophthalmic equipment using optical equipment. For example, as optical equipment for observing an eye, various types of equipment are used, such as an anterior ocular segment image acquiring apparatus, a fundus camera, and a scanning laser ophthalmoscope (SLO). In particular, an optical tomographic image acquiring apparatus (hereinafter referred to as “OCT apparatus”), which performs optical coherence tomography (OCT) utilizing an interference phenomenon of multi-wavelength light, is capable of obtaining a tomographic image of a sample with high resolution.

In the OCT apparatus, a sample is irradiated with measuring light being low-coherence light, and backward scattered light from the sample is measured through use of an interference system or an interference optical system to obtain depth-direction information on the sample. When the low-coherence light is increased in wavelength width, it is possible to obtain a tomographic image having a high resolution. In addition, the OCT apparatus scans the measuring light on the sample, to thereby be able to obtain a tomographic image in two dimensions including a scan direction and a depth direction. Therefore, the tomographic image of a retina on a fundus of an eye to be inspected can be acquired, and hence the OCT apparatus is widely used for ophthalmic diagnosis of a retina or the like.

Meanwhile, the OCT apparatus serving as ophthalmic equipment is generally mounted with an optical system for observing, for example, a fundus or an anterior eye in order to adjust alignment between the OCT apparatus and the eye to be inspected. As such an OCT apparatus, in Japanese Patent Application Laid-Open No. 2014-45906, there is disclosed an ophthalmic apparatus configured to determine a focusing position of an OCT optical system for acquiring a tomographic image based on a focusing position of a focus lens of an SLO optical system for fundus observation.

### SUMMARY OF THE INVENTION

A control apparatus for an optical tomographic image acquiring apparatus according to one embodiment of the present invention includes: a setting unit configured to set a focusing area on an object to be inspected; a condition acquiring unit configured to acquire a first focusing condition for a front image acquiring unit with respect to the focusing area, the focusing area being set in a partial region of a front image of the object to be inspected acquired by the front image acquiring unit, and being narrower than an image acquiring area of the front image; and a control unit configured to cause a tomographic image acquiring unit to acquire a tomographic image under a second focusing condition in accordance with the acquired first focusing condition.

Further features of the present invention will become apparent from the following description of exemplary embodiments with reference to the attached drawings.

### BRIEF DESCRIPTION OF THE DRAWINGS

FIG. 1 is a block diagram for illustrating a configuration of an optical tomographic image acquiring system according to a first embodiment of the present invention.

FIG. 2 is a diagram for illustrating an optical configuration of an OCT unit in the optical tomographic image acquiring system illustrated in FIG. 1.

FIG. 3 is a block diagram for illustrating a configuration of a control apparatus in the optical tomographic image acquiring system illustrated in FIG. 1.

FIG. 4 is a flow chart for illustrating a flow of control processing to be executed by the control apparatus in the first embodiment.

FIG. 5 is a diagram for exemplifying an operation screen to be displayed on a monitor by the control apparatus in the first embodiment.

FIG. 6 is a flow chart for illustrating a flow of control processing to be executed by the control apparatus in a second embodiment of the present invention.

FIG. 7 is a diagram for illustrating an example of a format for retaining information on the previous OCT image acquiring area, which is stored in a main storage apparatus, in the second embodiment.

FIG. 8 is a diagram for exemplifying an operation screen to be displayed on the monitor by the control apparatus in a third embodiment of the present invention.

FIG. 9 is flow chart for illustrating a flow of control processing to be executed by the control apparatus in the third embodiment.

FIG. 10 is a block diagram for illustrating a configuration of an optical tomographic image acquiring system in a fourth embodiment of the present invention.

FIG. 11 is a diagram for exemplifying an operation screen to be displayed on the monitor by the control apparatus in the fourth embodiment.

FIG. 12 is a flow chart for illustrating a flow of control processing to be executed by the control apparatus in the fourth embodiment.

FIG. 13 is a diagram for exemplifying an operation screen to be displayed on the monitor by the control apparatus in a fifth embodiment of the present invention.

### DESCRIPTION OF THE EMBODIMENTS

Preferred embodiments of the present invention will now be described in detail in accordance with the accompanying drawings.

In the apparatus described in Japanese Patent Application Laid-Open No. 2014-45906, a focus position of the OCT optical system is determined in conjunction with a focusing operation of a focus lens of an SLO optical system for fundus observation. A focusing position of the focus lens of the SLO optical system is obtained based on an image obtained by causing the SLO optical system to scan illumination light over the entire fundus of an eye to be inspected.

In recent years, in ophthalmic diagnosis or the like using an OCT apparatus, there has been a demand for the acquisition of a tomographic image in an extremely narrow region on the fundus in order to, for example, obtain information on a blood vessel. Meanwhile, in a technology disclosed in Japanese Patent Application Laid-Open No. 2014-45906, the fundus observation using the SLO optical system is performed on the entire area of the fundus of a wholly curved eye to be inspected, and hence the focusing position of the SLO optical system is also targeted on the entire area of the fundus. That is, a focused state is obtained with respect to a certain range in an optical axis direction, and hence the obtained focusing position does not so much take into consideration the influences of fundus shapes including a curve and irregularities. When a tomographic image is acquired from a partial region of the fundus, on the contrary, it is desired to achieve focusing with respect to an extremely narrow region in the optical axis direction in order to obtain intended tomographic information, if possible, in order to obtain a strictly focused state. Therefore, when the focusing position of the SLO optical system, at which the focusing has been determined to be achieved with respect to the entire fundus, is referred to in order to obtain the focusing position of the OCT optical system with respect to a partial region, it is required to take into consideration a difference between the desired focusing areas.

The present invention has been made in view of the above-mentioned circumstances, and provides a control apparatus configured to control the OCT apparatus so that a tomographic image can be acquired under an appropriate focusing condition by the OCT apparatus, an OCT system, a control method, and a program for executing the control method.

Now, exemplary embodiments for carrying out the present invention are described in detail with reference to the accompanying drawings. However, dimensions, materials, shapes, relative positions of components, and the like described in the following embodiment can be freely set, and can be changed depending on a configuration of an apparatus to which the present invention is applied or on various conditions. Throughout the drawings, like reference numerals are used to denote identical or functionally similar components.

### First Embodiment

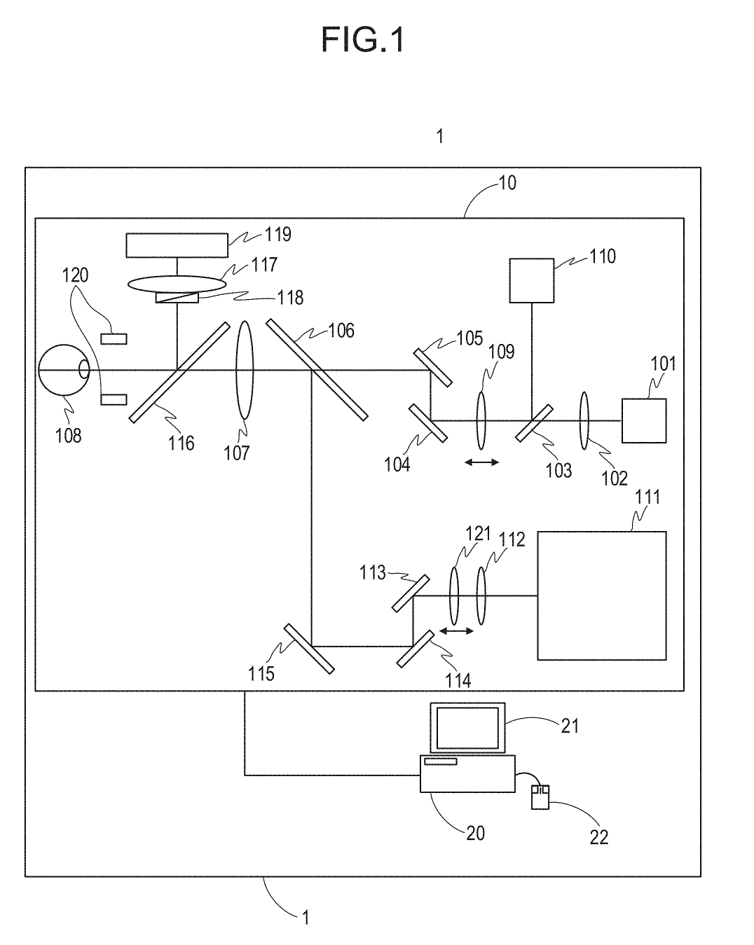

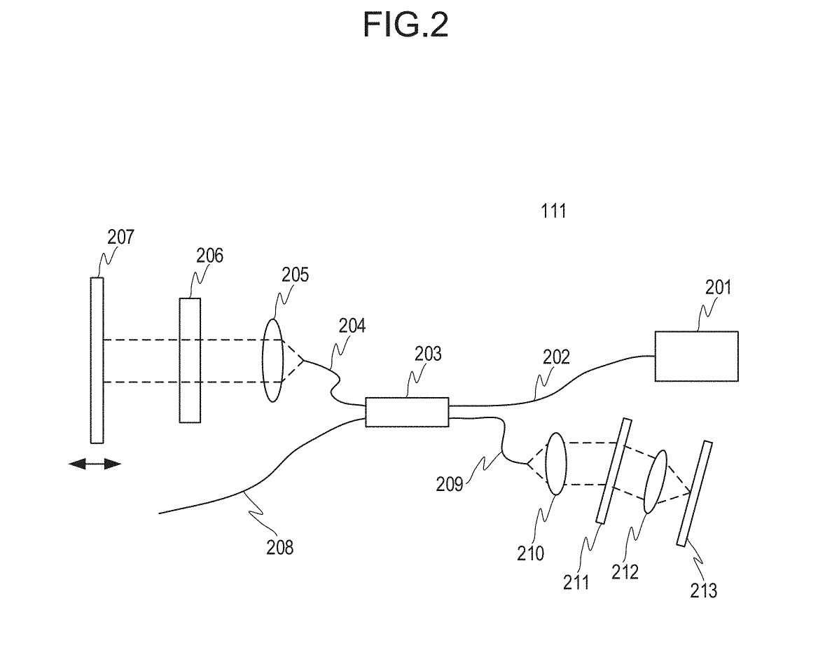

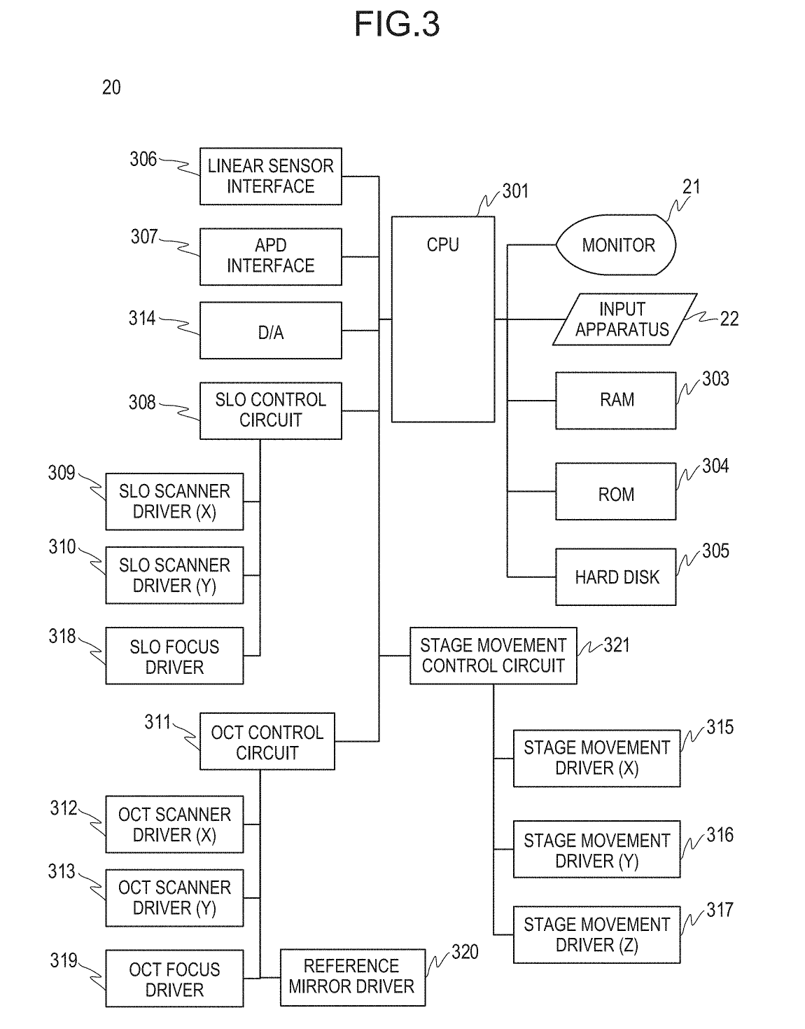

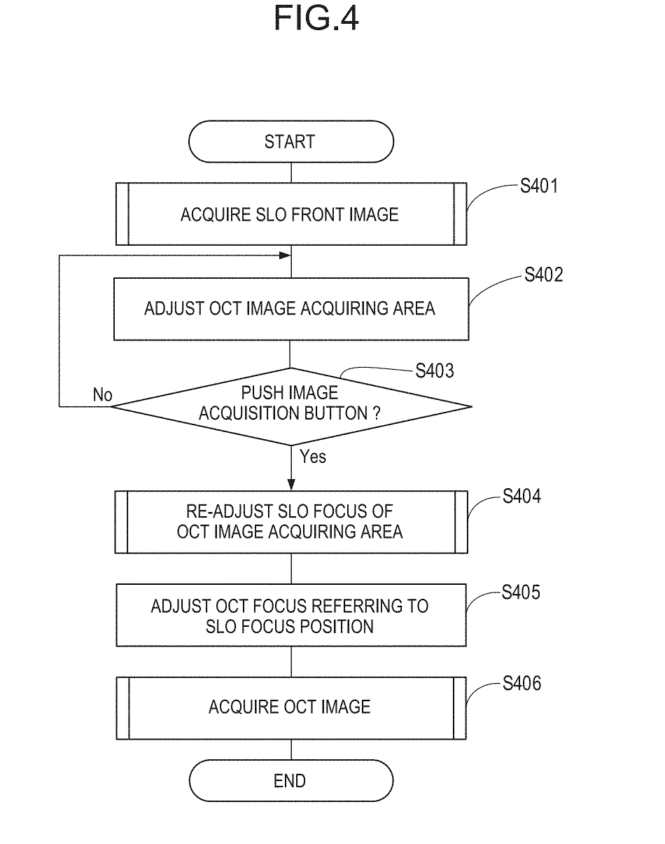

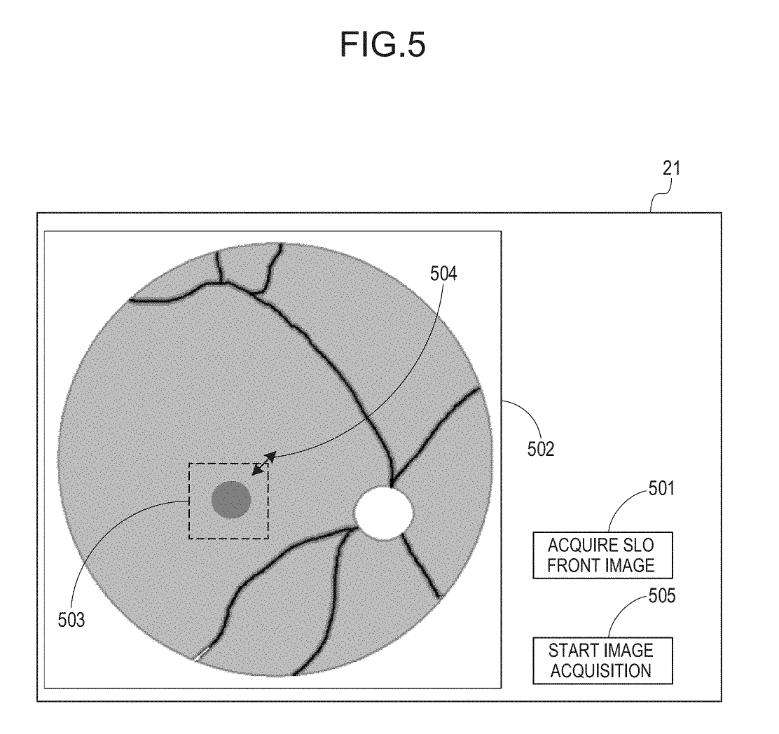

The description of the following embodiment is directed to an optical tomographic image acquiring system (OCT system) including a control apparatus and an optical tomographic image acquiring apparatus (OCT apparatus) to which the present invention is applied. FIG. 1 is a block diagram for illustrating an entire OCT system in a first embodiment of the present invention. FIG. 2 is a schematic diagram for illustrating an optical configuration of an OCT unit. FIG. 3 is a block diagram for illustrating the control apparatus. FIG. 4 is a flow chart for illustrating control processing to be executed by the control apparatus. FIG. 5 is a diagram for illustrating an example of a GUI screen to be displayed on a monitor by the control apparatus when a user designates an OCT image acquiring area on a fundus of an eye to be inspected in OCT image acquisition.

An optical tomographic image acquiring system 1 in the first embodiment includes an optical tomographic image acquiring apparatus 10 configured to acquire an image of an eye portion of a subject to be inspected (hereinafter referred to as “eye to be inspected”) as an image of an object to be inspected, and a control apparatus 20 configured to control the optical tomographic image acquiring apparatus 10 and execute image acquisition control processing on information obtained through the control. The optical tomographic image acquiring apparatus 10 in the first embodiment includes an optical tomographic image acquiring section (OCT image acquiring section) configured to acquire a tomographic image of (acquire tomographic information on) the fundus of the eye to be inspected, an anterior ocular segment image acquiring section configured to acquire an image of an anterior ocular segment of the eye to be inspected, and an SLO image acquiring section configured to acquire an image of the fundus of the eye to be inspected to obtain a fundus planar image. The optical tomographic image acquiring apparatus 10 is supported by a stage (not shown) movable in three directions, namely, an X-direction, a Y-direction, and a Z-direction. In FIG. 1, an ocular axis direction of an eye 108 to be inspected is defined as the Z-axis direction, a direction perpendicular to the drawing sheet and parallel to the fundus image of the eye 108 to be inspected is defined as the X-axis direction, and a direction perpendicular to the Z-axis and the X-axis is defined as the Y-axis direction.

A pair of LEDs 120 for illuminating the anterior ocular segment are arranged at positions symmetrical to each other with respect to the optical axis of measuring light (ocular axis) in front of the eye 108 to be inspected. A first beam splitter 116, an eyepiece lens (objective lens) 107, and a second beam splitter 106 are arranged on the optical axis of the measuring light in later stages of the pair of LEDs 120 for illuminating the anterior ocular segment. An optical path is branched off to the anterior ocular segment image acquiring section, the SLO image acquiring section, and the OCT image acquiring section, which are described above, by the above-mentioned beam splitters depending on the wavelength of target light. Now, configurations of the respective sections are described.

### Anterior Ocular Segment Image Acquiring Section

First, with reference to FIG. 1, the anterior ocular segment image acquiring section is described. The anterior ocular segment image acquiring section acquires an image of the anterior ocular segment of the eye 108 to be inspected, which is used for alignment between the eye 108 to be inspected and the optical tomographic image acquiring apparatus 10. The anterior ocular segment is illuminated by the pair of LEDs 120 for illuminating the anterior ocular segment, and reflected light from the anterior ocular segment is reflected toward a reflection direction of the first beam splitter 116. In the reflection direction of the first beam splitter 116, an image splitting prism 118, an anterior ocular segment focus lens 117, and an anterior ocular segment camera 119 are arranged in the stated order.

Anterior ocular segment reflected light, which is reflected by the first beam splitter 116, is formed into a split image by the image splitting prism 18 to be imaged on the anterior ocular segment camera 119 by the anterior ocular segment focus lens 117. The image of the anterior ocular segment, which is obtained through the imaging on the anterior ocular segment camera 119, is input to a CPU 301, which illustrated in FIG. 3, to be stored. Then, the alignment in the Z-axis direction is performed with reference to the split image of the anterior ocular segment, and the alignment in the X-direction and the alignment in the Y-axis direction are performed with reference to an amount of deviation between a pupil center in an anterior ocular segment image and the optical axis.

### SLO Image Acquiring Section

Next, the SLO image acquiring section (scanning laser eye inspecting section) being an apparatus for fundus observation is described. The SLO image acquiring section is arranged in the transmitting direction of the second beam splitter 106. The SLO image acquiring section includes an SLO scanner, an SLO focus lens 109, a holed mirror 103, a collimator lens 102, and a laser light source 101, which are arranged in the stated order from the second beam splitter 106. The SLO focus lens 109 is moved along the optical axis direction, which is indicated by the arrow in FIG. 1, by an SLO focus driver 318, which is described later with reference to FIG. 3, via a drive system (not shown). An avalanche photodiode (hereinafter referred to as “APD”) 110 is arranged in a reflection direction of the holed mirror 103.

The laser light source 101 can suitably use a semiconductor laser or a super luminescent diode (SLD) light source. In addition, in terms of the fundus observation, in order to alleviate glare felt by the subject to be inspected and maintain resolving power, laser light having a near-infrared wavelength range of from 700 nm to 1,000 nm can be suitably used. In the first embodiment, a semiconductor laser configured to emit laser light having a wavelength of 780 nm is used, but a light amount of the laser light can be changed by a control voltage.

The laser light emitted from the laser light source 101 is converted into a collimated beam by the collimator lens 102, and passes through a hole formed at the center of the holed mirror 103 to be guided to the SLO scanner through the SLO focus lens 109. The SLO scanner includes an SLO-X scanner 104 configured to scan the collimated beam in the X direction and an SLO-Y scanner 105 configured to scan the collimated beam in the Y direction. The collimated beam that has passed through the SLO scanner is further transmitted through the second beam splitter 106, passes through the eyepiece lens (objective lens) 107, and in transmitted through the first beam splitter 116 to enter the eye 108 to be inspected.

The collimated beam that has entered the eye 108 to be inspected applied as a spot-like beam to the fundus of the eye 108 to be inspected. At this time, the SLO focus lens 109 is moved to an appropriate position in the optical axis direction, to thereby achieve the focusing of the beam on the fundus. The focused beam is reflected or scattered by the fundus of the eye 108 to be inspected, and follows back the same optical path to return to the holed mirror 103. The reflected or scattered light is reflected by the holed mirror 103 to be received by the APD 110. A signal proportional to the intensity of reflection or scattering at an irradiation spot of the beam on the fundus is obtained from the APD 110. In addition, the SLO-X scanner 104 and the SLO-Y scanner 105 are operated to subject the beam to a raster scan on the fundus, to thereby be able to obtain a two-dimensional image of the fundus.

### OCT Unit

The optical tomographic image acquiring apparatus 10 includes the OCT image acquiring section including an OCT focus lens 121, a scanning part, an eyepiece part, and an OCT unit. The scanning part scans the measuring light for OCT, and the eyepiece part guides the measuring light to the eye 108 to be inspected. The OCT unit generates the measuring light, and obtains interference light from return light from the eye to be inspected to obtain an interference signal from the interference light. Now, an OCT unit 111 is described with reference to FIG. 2.

The OCT unit 111 splits low-coherence light into reference light and measuring light, superimposes the measuring light (return light), which has passed through the eye 108 to be inspected, and the reference light, which has passed through a reference object, one on another to generate the interference light, and spectrally disperses the superimposed light to output the interference signal. The interference signal is input to the CPU 301, and the CPU 301 analyzes the interference signal to form a tomographic image and a three-dimensional image of the fundus.

In more detail, the OCT unit 111 includes a low-coherence light source 201, an optical coupler 203, a reference optical system, a spectroscopic unit, and an optical fiber configured to allow those components to transmit light to one another. The low-coherence light source 201 is formed of a broadband light source configured to output the low-coherence light, and a super luminescent diode (SLD) is used as the broadband light source in the first embodiment. The low-coherence light includes light having a wavelength of a near-infrared region, and is light having a coherence length of about several tens of micrometers. For example, the low-coherence light has a wavelength within a range of from about 800 nm to about 900 nm. In addition, amplified spontaneous emission (AES) light source, an ultrashort pulse laser light source, for example, a titanium sapphire laser, or other such light source capable of emitting low-coherence light can also be used as the low-coherence light source 201.

The low-coherence light output from the low-coherence light source 201 is guided to the optical coupler 203 through an optical fiber 202. The optical fiber 202 is normally formed of a single-mode fiber. The optical coupler 203 splits the low-coherence light into reference light and measuring light. The reference light generated by the optical coupler 203 is guided to the reference optical system by an optical fiber 204.

The reference light emitted to the reference optical system by the optical fiber 204 is converted into a collimated light flux by a collimator lens 205, and the collimated light flux then passes through a glass block 206 serving as a dispersion compensation unit used for matching the dispersion characteristics of the reference light and the return light. After that, the collimated light flux is reflected by a reference mirror 207. The reflected reference light passes through the same optical path to enter the optical fiber 204. The reference mirror 207 is movable in a traveling direction of the reference light, and can adjust an optical path length of the reference light through the movement. The optical path length by which the measuring light passes (measuring light path length) changes depending on, for example, an ocular axis length of the eye 108 to be inspected and a distance between the eyepiece lens (objective lens) 107 and the eye 108 to be inspected, but it is possible to match the optical path lengths of the reference light and the measuring light by adjusting the position of the reference mirror 207. When the reference light and the measuring light are combined after the optical path lengths thereof are matched, interference in accordance with a tissue of the fundus in a depth direction is obtained.

Meanwhile, the measuring light generated by the optical coupler 203 is guided to the OCT focus lens 121, the scanning part, and the eyepiece part, which are described above, by an optical fiber 208. The OCT focus lens 121 is moved along the optical axis direction, which is indicated by the arrow in FIG. 1, by an OCT focus driver 319, which is described later, via a drive system (not shown). The scanning part includes an OCT scanner illustrated in FIG. 1, and the eyepiece part includes the eyepiece lens 107. The measuring light guided to the eye 108 to be inspected via the scanning part and the eyepiece part is reflected and scattered by a retina of the eye to be inspected, and is then input again to the optical fiber 208 as return light. The return light guided into the optical couple 203 via the optical fiber 208 is multiplexed with the reference light as interference light, and passes through an optical fiber 209 to be guided to the spectroscopic unit. The interference light emitted to the spectroscopic unit by the optical fiber 209 is converted into collimated light by a collimator lens 210, and is then spectrally dispersed by a diffraction grating 211 on a wavelength-to-wavelength basis. The spectrally dispersed light having the respective wavelengths is imaged on a linear sensor 213 by a focus lens 212 for spectral dispersion. For example, a CCD sensor or a CMOS sensor can be used as the linear sensor 213. With this configuration, it is possible to obtain the interference signal, which is obtained by spectrally dispersing the interference light, from the linear sensor 213.

### OCT Focus Lens, Scanning Part, and Eyepiece Part

Next, the OCT focus lens 121, the scanning part, and the eyepiece part are described with reference to FIG. 1. The measuring light generated by the OCT unit 111 is converted into collimated light by a collimator lens 112, and passes through an OCT-X scanner 113 and an OCT-Y scanner 114 via the OCT focus lens 121. The measuring light that has passed through those scanners is then reflected by a mirror 115 and the second beam splitter 106, and passes through the eyepiece lens (objective lens) 107 to enter the eye 108 to be inspected. At this time, the OCT focus lens 121 is moved to an appropriate position in the optical axis direction, to thereby achieve the focusing of the measuring light on the fundus. In the same manner as in the case of the laser light in the SLO image acquiring section, the measuring light that has entered the eye 108 to be inspected is reflected and scattered by the fundus, and follows back the same optical path to return to the OCT unit 111. By two-dimensionally scanning the measuring light on the fundus through use of those scanners, it is possible to acquire three-dimensional tomographic information (three-dimensional image) from the fundus.

### Controller

Next, the control apparatus 20 configured to control the optical tomographic image acquiring apparatus 10 is described with reference to FIG 3. A central processing unit (CPU 301) included in the control apparatus 20 is connected to a monitor 21, an input apparatus 22, a main storage apparatus (RAM 303), a storage apparatus (ROM 304), and a hard disk drive 305. In response to instructions for display control issued by the CPU 301, the monitor 21 displays, for example, the two-dimensional image of the fundus, the tomographic image obtained by the OCT image acquiring section, different kinds of data including patient information, different kinds of images for alignment, and a GUI. A mouse, a keyboard, a GUI, and the like are used as the input apparatus 22. The storage apparatus (ROM 304) stores a program for executing processing illustrated in the flow chart of FIG. 4. The input apparatus 22 is also used when, for example, the user designates an image acquiring area on the fundus of the eye 108 to be inspected, which is to be used by the optical tomographic image acquiring apparatus 10.

The CPU 301 is also connected to a linear sensor interface 306, an APD interface 307, and a D/A converter 314. The linear sensor interface 306 receives data on the linear sensor 213, which is the output from the OCT image acquiring section. The APD interface 307 receives data on the APD 110, which is the output from the SLO image acquiring section. The D/A converter 314 generates a voltage for controlling the intensity of the laser light emitted by the laser light source 101.

In order to control different kinds of components of the optical tomographic image acquiring apparatus 10, the CPU 301 is further connected to an SLO control circuit 308, an OCT control circuit 311, and a stage movement control circuit 321. With this configuration, the control apparatus 20 adjusts the positions or settings of different kinds of members of the optical tomographic image acquiring apparatus 10. Specifically, the control apparatus 20 transmits a command for instructing the adjustment to the optical tomographic image acquiring apparatus 10 along with a control parameter, to thereby adjust the positions or settings of different kinds of optical members of the optical tomographic image acquiring apparatus 10.

In more detail, the SLO control circuit 308 uses an SLO scanner driver (X) 309 and an SLO scanner driver (Y) 310 to control the corresponding SLO-X scanner 104 and the corresponding SLO-Y scanner 105, respectively. Specifically, the SLO control circuit 308 controls the scan center position, scan width, and scan rate of each scanner in response to an instruction from the CPU 301. At the same time, the CPU 301 is allowed to obtain the scan position of a beam used for the scan from the SLO control circuit 308. The SLO control circuit 308 further moves the position of the SLO focus lens 109 on the optical axis by the SLO focus driver 318, to thereby achieve the focusing of the scanned beam on the fundus.

The OCT control circuit 311 uses an OCT scanner driver (X) 312 and an OCT scanner drives (Y) 313 to control the corresponding OCT-X scanner 113 and the corresponding OCT-Y scanner 114, respectively. Specifically, the OCT control circuit 311 controls the scan center position, scan width, and scan rate of each scanner in response to an instruction from the CPU 301. At the same time, the CPU 301 is allowed to obtain the scan position of the measuring light from the OCT control circuit 311. The OCT control circuit 311 further moves the position of the OCT focus lens 121 on the optical axis by the OCT focus driver 319, to thereby achieve the focusing of the measuring light on the fundus. In addition, the OCT control circuit 311 moves the reference mirror 207 along the optical axis direction by a reference mirror driver 320 via a drive system (not shown), to thereby adjust the optical path length of the reference light.

The stage movement control circuit 321 controls a stage movement driver (X) 315, a stage movement driver (Y) 316, and a stage movement driver (Z) 317. Those drivers can operate drive systems (not shown) corresponding to the X-axis direction, the Y-axis direction, and the Z-axis direction, which are configured to support the optical tomographic image acquiring apparatus 10 so as to be movable in the X-axis direction, the Y-axis direction, and the Z-axis direction. A stage configured to support the optical tomographic image acquiring apparatus 10 so as to be movable is arranged on a base portion (not shown), and moves the optical tomographic image acquiring apparatus 10 on the stage relative to be base portion. Through the operation of the stage, the optical tomographic image acquiring apparatus 10 and the eye 108 to be inspected are aligned with each other.

The CPU 301 uses the program stored in a program storage ROM (ROM 304) to execute the control processing described later with reference to the flow chart of FIG. 4, to thereby control the optical tomographic image acquiring apparatus 10 to acquire a suitable tomographic image of the eye 108 to be inspected. At that time, the CPU 301 executes the above-mentioned program, to thereby set the image acquiring area to be used by OCT based on an operation performed on the input apparatus 22 by the user. The CPU 301 or the above-mentioned circuits, drivers, and the like carry out the control of the alignment, which is the adjustment of the relative positions of the fundus of the eye 108 to be inspected, which is the object to be inspected, and the optical tomographic image acquiring apparatus 10, and carry out the focusing processing of the SLO image acquiring section and the OCT image acquiring section.

### Alignment Adjustment

The above-mentioned alignment adjustment carried out by the optical tomographic image acquiring apparatus 10 is described. In the alignment adjustment, a pupil is detected from the image of the anterior ocular segment acquired by the anterior ocular segment camera 119, and the stage is driven in the X-axis direction and the Y-axis direction by the stage movement control circuit 321 so that the center of the pupil matches the center of the image. With reference to the image of the anterior ocular segment, which has been split into, for example, an upper image and a lower image by the image splitting prism 118, the stage is driven in the Z-axis direction by the stage movement control circuit 321 so as to correct a deviation between the upper image and the lower image. Through the above-mentioned drive of the stage, it is possible to adjust the alignment of the optical tomographic image acquiring apparatus 10 with respect to the eye 108 to be inspected.

### Fundus Observation by SLO, Confirmation of OCT Image Acquisition Position, and Focus Adjustment

Next, processing for acquiring a fundus image by the SLO image acquiring section is described as well as confirmation of the position for the OCT image acquisition and focus adjustment for the OCT image acquisition, which are performed through use of the fundus image.

In the fundus observation using the SLO image acquiring section, the CPU 301 inputs a default value to the D/A converter 314, and causes the laser light source 101 to emit the laser light. The CPU 301 further sets, in the SLO control circuit 308, a default X-scan center position, a default scan speed, a default scan width in the X-axis direction for the SLO scanner driver (X) 309. At the same time, the CPU 301 also sets, in the SLO control circuit 308, a default Y-scan center position, a default scan speed, a default scan width in the Y-axis direction for the SLO scanner driver (Y) 310. With those settings, a region on the retina over which a beam is scanned by the SLO image acquiring section is set. Through the scanning of the beam, a signal proportional to the intensities of the reflection and scattering of the beam on the retina is output from the APD 110.

The CPU 301 obtains a scanning position of the beam on the fundus based on positional information on the scanner obtained from the SLO control circuit 308. A signal intensity obtained by the APD 110 superimposed on the obtained scanning position, to thereby be able to obtain a retina image. The obtained retina image is displayed on the monitor 21 as a two-dimensional planar image by the CPU 301 being a display control unit. The user is allowed to refer to the two-dimensional planar image to confirm a position or an area (hereinafter referred to as “OCT image acquiring area”) for acquiring the three-dimensional tomographic information by OCT. In addition, the position of the SLO focus lens 109 on the optical axis is controlled so as to maximize the contrast of the two-dimensional planar image, to thereby allow the SLO image acquiring section to achieve the focusing on the fundus.

At this time, the scan width in the X-axis direction and the scan width in the Y-axis direction of the beam to be used by the SLO scanner are caused to match the OCT image acquiring area designated by the user through the operation performed on the input apparatus 22. Under this state, the position of the SLO focus lens 109 on the optical axis is controlled via the SLO focus driver 318 so as to maximize the contrast of the retina image to be obtained, to thereby allow the focus of the SLO image acquiring section to be achieved in a desired OCT image acquiring area.

The OCT focus lens 121 of the OCT image acquiring section and the SLO focus lens 109 of the SLO image acquiring section are arranged in separate optical systems. In the first embodiment, those focus lenses are driven in conjunction with each other, and relationships between the focus positions and driving amounts of the respective focus lenses are stored in the hard disk drive 305 as table information. When the table information is used to drive the OCT focus driver 319 in conjunction with the SLO focus driver 318, the focus of the OCT image acquiring section can be achieved simultaneously while the focus of the SLO image acquiring section is achieved.

### User Operation

FIG. 5 is a diagram for illustrating an example of an operation screen to be displayed on the monitor 21 at the time of acquiring an OCT image. On the operation screen, an SLO front image acquiring button 501, an SLO retinal surface image 502, and an image acquisition start button 505 are displayed. In the SLO retinal surface image 502, a region display frame 503 and a cursor 504 are superimposed to be displayed. The SLO front image acquiring button 501 is a button for starting processing for causing the SLO image acquiring section to acquire a front image of a retina. When the SLO front image acquiring button 501 is pushed, a default scan center position and default scan widths in the X-axis direction and the Y-axis direction are set in the SLO control circuit 308 to scan the beam on the retina and achieve the focus of the SLO focus lens 109. Specifically, when the user uses the mouse to place a pointer of a cursor over the button and perform a click operation, the CPU 301 receives the pushing operation of the button. After the above-mentioned processing, the SLO retinal surface image 502 generated by the CPU 301 through use of luminance information acquired from a default XY region is displayed on the monitor 21.

The region display frame 503 for indicating the OCT image acquiring area on the fundus, which is set through use of the input apparatus 22, is displayed together over the displayed SLO retinal surface image 502. In addition, the cursor 504 for changing the OCT image acquiring area by changing the size, aspect ratio, or the like of the region display frame 503 is displayed along with the region display frame 503. The user is allowed to use the cursor 504 through use of the input apparatus 22 to change the display position, size, and the like of the region display frame 503. The CPU 301 supplies the OCT control circuit 311 with setting values for the OCT scanner driver (X) 312 and the OCT scanner driver (Y) 313 in accordance with the display position. After that, when the image acquisition start button 505 is pushed, the CPU 301 performs focus adjustment on the measuring light with respect to the OCT image acquiring area designated via the OCT focus driver 319, and acquiring the tomographic image of the retina within the image acquiring area.

The above-mentioned acquisition processing for the three-dimensional tomographic information within the designated image acquiring area is described in more detail with reference to the flow chart of FIG. 4. First, when the SLO front image acquiring button 501 on the operation screen is pushed, image acquisition processing for the tomographic image is started by the OCT image acquiring section. When the image acquisition processing is started, the CPU 301 executes the acquisition processing for an SLO front image in Step S401. In the first embodiment, before the execution of the processing of Step S401, the alignment between the eye 108 to be inspected and the optical tomographic image acquiring apparatus 10 is completed in advance through use of the anterior ocular segment image. In SLO front image acquiring processing, an SLO retinal surface image is acquiring by scanning a beam on the retina over an area having a default scan center position and default scan widths in the X-axis direction and the Y-axis direction. In regard to the SLO retinal surface image, the focused state is confirmed based on a contrast or the like to repeatedly perform the focus adjustment, the acquisition of the SLO retinal surface image, and contrast evaluation. When it is confirmed that the SLO retinal surface image has been obtained under the focused state, the obtained image is displayed on the monitor 21 as the SLO retinal surface image 502.

When the SLO retinal surface image 502 is displayed on the operation screen of the monitor 21, the flow advances to Step S402, and the adjustment or setting of the OCT image acquiring area is performed through use of the cursor 504. Specifically, the OCT image acquiring area is adjusted by the user moving the cursor 504 through use of the input apparatus 22. When the operation for moving the cursor 504 or other such operation is temporality stopped, the flow advances to Step S403, and the CPU 301 determines whether or not the image acquisition start button 505 has been pushed. When the CPU 301 determines that the image acquisition start button 505 has not been pushed, the flow returns to Step S402 to repeatedly perform the subsequent processing steps. When the CPU 301 determines in Step S403 that the image acquisition start button 505 has been pushed, the flow advances to Step S404.

In Step S404, the CPU 301 performs the focusing processing on the SLO focus lens 109 with respect to the OCT image acquiring area adjusted or set on the SLO retinal surface image 502 by the region display frame 503. In the first embodiment, the SLO focus lens 109 is moved along the optical axis via the SLO focus driver 318 so as to maximize the contrast of the SLO front image in the OCT image acquiring area. The movement of the SLO focus lens 109, the acquisition of the SLO retinal surface image, and the contrast evaluation are repeatedly performed until the focused state is obtained. When the focused state is obtained, the flow advanced to Step S405.

In Step S405, the CPU 301 moves the OCT focus lens 121 along the optical axis based on the position of the SLO focus lens 109 on the optical axis. The driving amount and position on the optical axis of the SLO focus lens 109 and the driving amount and position on the optical axis of the OCT focus lens 121 are associated with each other to be stored in advance in the hard disk drive 305 as the table information as described above. The CPU 301 uses the table information to drive the OCT focus lens 121 via the OCT focus driver 319. After the driving of the OCT focus lens 121 is completed, the flow advances to Step S406.

In Step S406, the CPU 301 performs the image acquisition processing for a retina tomographic image (three-dimensional tomographic information) in an OCT image acquiring area designated by the OCT image acquiring section. In the image acquisition processing, processing for scanning the measuring light over the OCT image acquiring area, processing for generating interference light between the return light from the OCT image acquiring area and the reference light, processing for sampling an interference signal from the interference light, processing for generating three-dimensional luminance information from the interference signal, and processing for generating a three-dimensional tomographic image from the luminance information are performed. Although not shown herein, processing for adjusting the optical path length of the reference light may be performed along with the above-mentioned processing.

Through the execution of the above-mentioned processing (or steps), it is possible to acquire focusing information on the SLO focus lens 109 within a narrow focusing area in the optical axis direction in accordance with the OCT image acquiring area. In addition, when a focus position for OCT is adjusted through use of the focusing information on the SLO focus lens 109, the retina tomographic image can be acquired at the position of the OCT focus lens 121 appropriate for the OCT image acquiring area.

The relationship of arrangement between the anterior ocular segment image acquiring section, the SLO image acquiring section, and the OCT image acquiring section, which are described above, and the beam splitters configured to branch the optical path off to the respective sections is merely an example, and is not limited to the arrangement to which the present invention is to be applied. For example, the SLO image acquiring section and the OCT image acquiring section may be arranged in a rejection direction of the first beam splitter 116 with the anterior ocular segment image acquiring section being arranged in the transmitting direction, or the SLO image acquiring section may be arranged in the reflection direction of the second beam splitter 106 with the OCT image acquiring section being arranged in the transmitting direction. That is, the arrangement of the optical members and the respective image acquiring sections can be changed appropriately depending on space or the like allowed in a casing at the time of constructing the apparatus. In addition, the scanner is not limited to the scanner corresponding to each of the X-axis direction and the Y-axis direction. For example, a scanner capable of scanning light beams in the two directions may be used.

As described above, the control apparatus 20 according to the first embodiment includes a setting unit, a condition acquiring unit, and a control unit. The setting unit sets a focusing area based on an instruction input by the input apparatus 22 exemplified by the cursor 504. The condition acquiring unit is exemplified by the SLO control circuit 308, and the control unit is exemplified by the OCT control circuit 311. As an example of the setting unit, in the first embodiment, the CPU 301 executes the above-mentioned processing. The control apparatus 20 described above executes a control method of performing the respective steps of the flow chart illustrated in FIG. 4 by those respective units. In this case, the CPU 301 serves as the setting unit to set the region on the fundus of the eye 108 to be inspected, which is designated by the region display frame 503 through use of the cursor 504, as the focusing area. The SLO control circuit 308 acquires the front image of the fundus by the SLO image acquiring section serving as a front image acquiring unit. Then, a first focusing condition for the region display frame 503, which is the focusing area set in an initial image acquiring area of a retinal surface image being the front image, is acquired. At that time, the focusing area set in the image acquiring area for the SLO image acquiring section configured to acquire the front image of the fundus is narrower than the image acquiring area. The OCT control circuit 311 controls the tomographic image acquiring section to acquire a tomographic image under a second focusing condition for an OCT controller in accordance with the acquired first focusing condition. It is preferred that the front image of the fundus for setting the focusing area be acquired under the focused state with respect to the first image acquiring area used when the SLO image acquiring section acquires the front image.

The CPU 301 exemplifying the display control unit included in the control apparatus 20 causes the monitor 21 exemplifying a display unit to display the SLO retinal surface image 502. In this case, the display control unit causes the monitor 21 to superimpose the focusing area displayed by the region display frame 503 or the image acquiring area on the displayed SLO retinal surface image to display the SLO retinal surface image 502 with the focusing area or the image acquiring area. The focusing area or the image acquiring area is displayed as the region display frame 503 exemplifying a display form for indicating a predetermined region of the displayed front image. With the region display frame 503, it is possible to change the focusing area by performing at least one of the movement or deformation through use of the cursor 504.

In the first embodiment, the set focusing area has the same area as the image acquiring area for acquiring the tomographic image. However, as described later, the focusing area and the image acquiring area may differ from each other. Further, in the firs embodiment, as an example of the front image acquiring unit, the SLO image acquiring section configured to acquire the image of the surface of the object to be inspected (retinal surface) through use of reflected/scattered light of the laser light scanned on the object to be inspected is used. Further, a tomographic image acquiring unit is exemplified by the OCT image acquiring section configured to acquire a tomographic image through use of the interference light obtained by multiplexing the return light of the scanned measuring light from the object to be inspected (fundus) and the reference light corresponding to the return light with each other. However, the configurations of the image acquiring units are not limited to the configurations of those examples as long as the first focusing condition for the front image acquiring unit and the second focusing condition for the tomographic image acquiring unit are associated with each other and the tomographic image can be acquired under the second focusing condition in accordance with the first focusing condition as described later. As described later, a fundus camera or other such component configured to obtain a fundus planar image can be employed as the from image acquiring unit, and a component other than the OCT image acquiring section can also be used as the tomographic image acquiring unit as long as the component can acquire a tomographic image.

### Second Embodiment

In the above-mentioned first embodiment, the processing for adjusting the focusing position of the SLO focus lens 109 and the focusing position of the OCT focus lens 121 in conjunction therewith is performed when the image acquisition start button 505 is pushed. However, there may be no difference between the respective positions in a plurality of tomographic image acquiring areas for which a plurality of tomographic images are obtained, for example, the tomographic images of a plurality of adjacent regions of the same eye to be inspected are acquired. In such a case, there is no great change in focusing positions of the SLO focus lens 109 and the OCT focus lens 121, and hence it is also possible to employ a configuration in which re-adjustment processing for those focusing positions is omitted to reduce a processing time period.

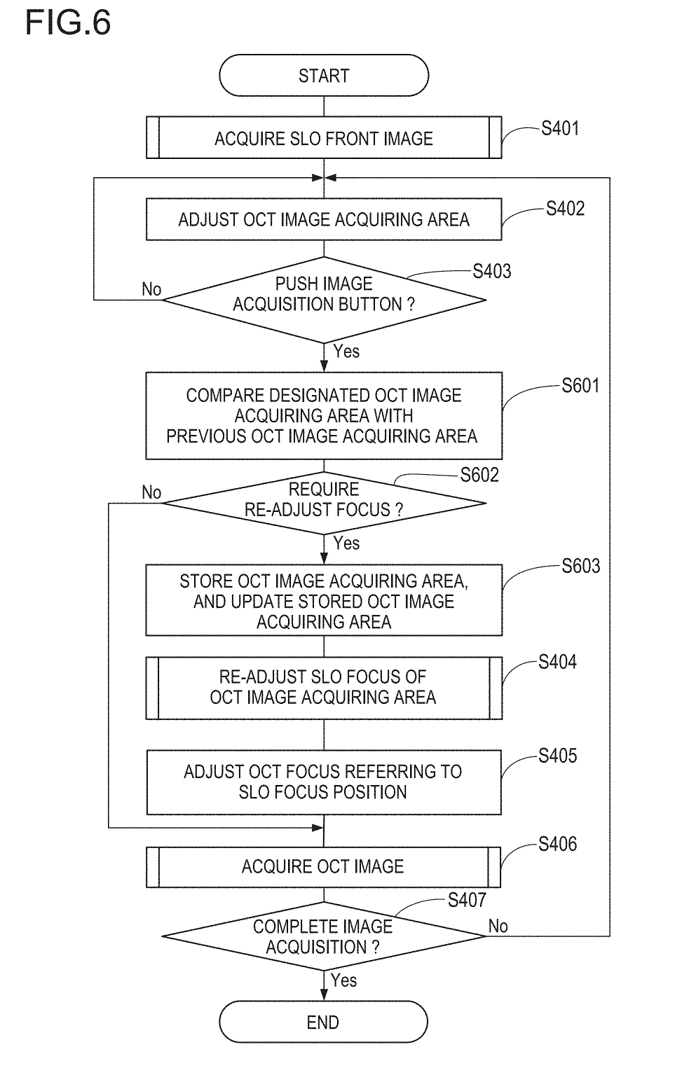

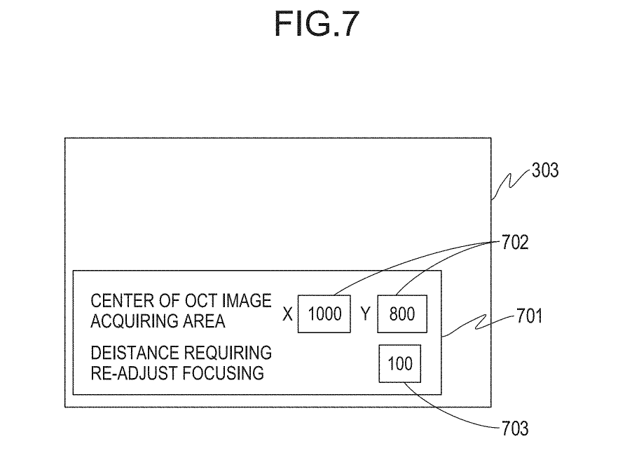

Now, with reference to FIG. 6 and FIG. 7, processing for determining whether or not it is required to re-adjust the focusing position of the OCT focus lens 121 based on the OCT image acquiring area in this manner is described. In a second embodiment of the present invention, the same processing steps as the processing steps described in the above-mentioned first embodiment are denoted by the same reference symbols (step numbers), and detailed descriptions thereof are omitted below.

In the second embodiment, when the SLO front image acquiring button 501 on the operation screen is pushed, OCT image acquisition processing is started, and the CPU 301 executes the acquisition processing for the SLO retinal surface image 502 in Step S401. When the SLO retinal surface image 502 is displayed on the monitor 21, the adjustment or setting of the image acquiring area for OCT is performed in Step S402, and the CPU 301 subsequently determines in Step S403 whether or not the image acquisition start button 505 has been pushed. When the CPU 301 determines that the image acquisition start button 505 has not been pushed, Step S402 and the subsequent processing steps are repeatedly performed.

When the CPU 301 determines in Step S403 that the image acquisition start button 505 has been pushed, the flow advances to Step S601. In Step S601, the CPU 301 performs processing for comparing the set OCT image acquiring area with the previous OCT image acquiring area in terms of the display position in the SLO retinal surface image 502. An example of the information obtained at the time of the previous image acquisition to be used for the processing for the comparison is illustrated in FIG. 7. The information includes X and Y coordinates 702 of the scan center of the previous OCT image acquiring area and a threshold distance 703 between image acquiring areas requiring focus re-adjustment. Those pieces of information are stored in the RAM 303 connected to the CPU 301. A value determined in advance is set as the threshold distance 703 requiring the focus re-adjustment, or the user may be allowed to appropriately set the threshold distance 703.

In the actual processing for the comparison, a distance between the center positions is calculated from the X and Y coordinates 702 of the center of the previous OCT image acquiring area and the X and Y coordinates of the scan center of the image acquiring area for OCT set in Step S402. The CPU 301 determines whether or not the focus re-adjustment is required based on whether or not the calculated distance exceeds the threshold distance 703 requiring the re-adjustment of the focusing position (Step S602). When the CPU 301 determines in Step S602 that the re-adjustment is not required due to the distance between the center positions being equal to or smaller than a threshold value, the flow advances to Step S406, and the OCT image acquiring section acquires the retina tomographic image. Subsequently, it is determined in Step S407 whether or not the image acquisition is to be completed. When the image acquisition is not to be completed, the flow returns to Step S402, and the CPU 301 continues to perform the subsequent processing steps. When the CPU 301 determines in Step S602 that the re-adjustment is required, the flow advances to Step S603.

In Step S603, the CPU 301 updates the X and Y coordinates 702 of the scan center of the previous OCT image acquiring area with information on the current OCT image acquiring area. Subsequently in Step S404, the CPU 301 controls the position of the SLO focus lens 109 on the optical axis so as to maximize the contrast of the SLO retinal surface image in the current OCT image acquiring area. In accordance with the movement of the SLO focus lens 109 to the focusing position, in Step S405, the CPU 301 moves the OCT focus lens 121 along the optical axis based on the position of the SLO focus lens 109 on the optical axis. After that, the CPU 301 carries out Step S406 and the subsequent processing steps.

In the second embodiment, the CPU 301 further serves as a determination unit configured to determine, when a tomographic image is acquired plural times, whether or not the distance between the focusing areas set with respect to the tomographic images exceeds a threshold value. When the determination unit determines that the distance between the focusing areas is equal to or smaller than the threshold value, the OCT control circuit avoids changing the above-mentioned second focusing condition for the OCT image acquiring section when the OCT image acquiring section acquires the retina tomographic image. When the distance between the focusing areas exceeds the threshold value, the second focusing condition is changed in accordance with the above-mentioned processing.

Through the execution of the above-mentioned processing, the re-adjustment processing for the focusing position of the SLO image acquiring section is not performed when the distance between the center of the previous OCT image acquiring area and the center of the current OCT image acquiring area is equal to or smaller than the set distance. Therefore, a time period for image acquisition can be reduced to a lower level than in the case of always performing the re-adjustment processing for the focusing position of the OCT image acquiring section subsequently to the re-adjustment processing for the focusing position of the SLO image acquiring section at the time of the image acquisition. As a result, it is also expected to improve, for example, the user's convenience compared with the above-mentioned first embodiment.

### Third Embodiment

There is known a technology called OCT angiography (hereinafter referred to as “OCTA”) for superimposing a plurality of tomographic images obtained through OCT one on another and extracting a part exhibiting a large temporal change, to thereby visualize a state of a blood flow on the retina. In image acquisition for performing OCTA (hereinafter referred to as “OCTA image acquisition”), an image in a narrower region is generally acquired than at the time of the acquisition of the tomographic image through OCT. Therefore, it is also possible to employ a configuration in which an OCT focus adjustment region to be caused to correspond to the SLO image acquiring season is switched between the OCT image acquisition and the OCTA image acquisition so as to reflect a difference in image acquiring area between the OCT image acquisition and the OCTA image acquisition.

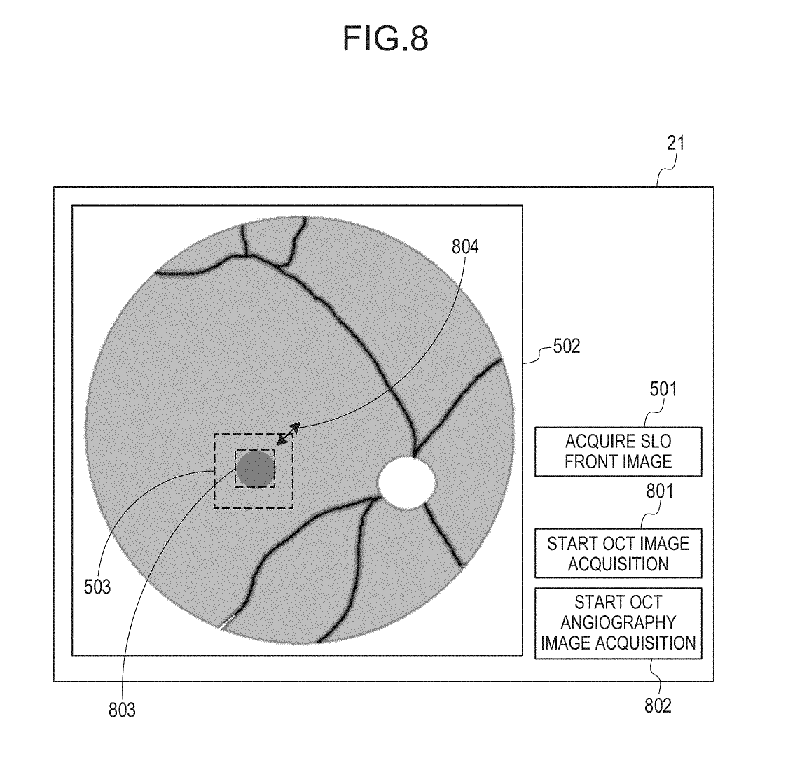

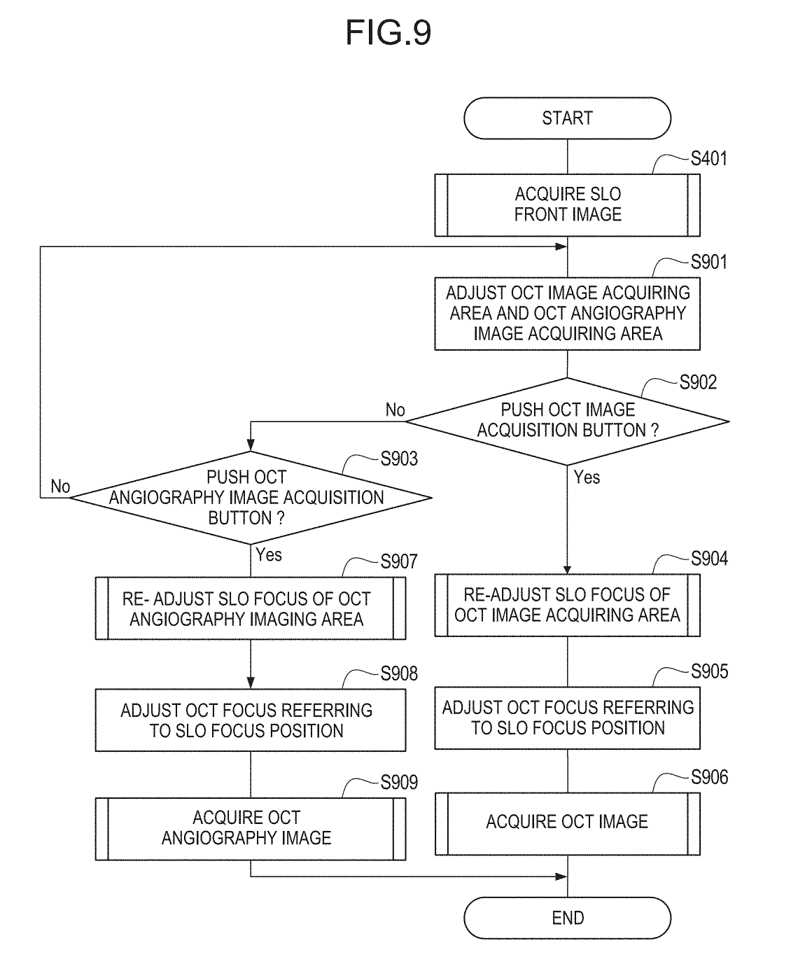

Now, with reference to FIG. 8 and FIG. 9, processing for changing focus adjustment region between the OCT image acquisition and the OCTA image acquisition in this manner is described. In a third embodiment of the present invention, the same processing steps as the processing steps described in the above-mentioned first or second embodiment are denoted by the same reference symbols (step numbers), and detailed descriptions thereof are omitted below.

FIG. 8 is a diagram for illustrating an example of an operation screen to be displayed on the monitor 21. On the operation screen, an OCT image acquisition start button 801 and an OCTA image acquisition start button 802 are displayed in addition to the SLO front image acquiring button 501 and the SLO retinal surface image 502, which are described above. In addition, on the displayed SLO retinal surface image 502, a cursor 804 and a second region display frame 803 for indicating an OCTA image acquiring area are displayed together in addition to the region display frame 503 described above.

The region display frame 503 indicates the OCT image acquiring area set through use of the input apparatus 22, and the second region display frame 803 indicates the OCTA image acquiring area set through use of the input apparatus 22. The cursor 804 in the third embodiment is used for changing the OCT image acquiring area and the OCTA image acquiring area. The user uses the input apparatus 22 to point at the boundary of the region display frame 503 with the cursor 804 and move the boundary, to thereby move and change the OCT image acquiring area. Then, through the pointing and movement of the boundary of the second region display frame 803, the OCTA image acquiring area is moved and changed. When the OCT image acquisition start button 801 is pushed, the focus adjustment is carried out for the SLO image acquiring section and for the OCT image acquiring section in accordance therewith, with respect to the OCT image acquiring area. Then, the OCT image is acquired. In addition, when the OCTA image acquisition start button 802 is pushed, the focus adjustment is carried out for the SLO image acquiring section and for the OCT image acquiring section in accordance therewith, with respect to the OCTA image acquiring area. Then, an OCTA image is acquired.

Processing for performing the OCT image acquisition or the OCTA image acquisition with the above-mentioned display is described with reference to a flow chart of FIG. 9. When the SLO front image acquiring button 501 on the operation screen is pushed, the image acquisition processing for the tomographic image is started, and the CPU 301 executes the acquisition processing for SLO front image in Step S401. When the SLO retinal surface image 502 is displayed on the monitor 21, the adjustment or setting of the image acquiring area for OCT or OCTA is performed in Step S901. As described above, the image acquiring area for OCT or OCTA is adjusted by the user with the cursor 504 through use of the input apparatus 22.

In Step S902, the CPU 301 determines whether or not the OCT image acquisition start button 801 has been pushed. When the CPU 301 determines that the OCT image acquisition start button 801 has not been pushed, the flow advances to Step S903, in which the CPU 301 determines whether or not the OCTA image acquisition start button 802 has been pushed. When the CPU 301 determine in Step S903 that the OCTA image acquisition start button 802 has not been pushed, the flow returns to Step S901 to repeatedly perform the subsequent processing steps.

When the CPU 301 determines in Step S902 that the OCT image acquisition start button 801 has been pushed, the flow advances to Step S904. In Step S904, the CPU 301 controls the position of the SLO focus lens 109 on the optical axis so as to maximize the contrast of the SLO retinal surface image with respect to the image within the region display frame 503. That is, in the same manner as in Step S404 in the first embodiment, the focusing operation of the SLO image acquiring section is performed with respect to the OCT image acquiring area. After the focusing operation is completed, subsequently in Step S905, in the same manner as in Step S405, the CPU 301 moves the OCT focus lens 121 along the optical axis based on the position of the SLO focus lens 109 on the optical axis. After the OCT focus lens 121 is stopped on the optical axis, in Step S906, in the same manner as in Step S406, the OCT image acquiring section executes the image acquisition for the retina tomographic image in the OCT image acquiring area.

When the CPU 301 determines in Step S903 that the OCTA image acquisition start button 802 has been pushed, the flow advances to Step S907. In Step S907, the CPU 301 control the position of the SLO focus lens 109 on the optical axis so as to maximize the contrast of the SLO retinal surface image with respect to the image within the second region display frame 803. That is, a target area for the focusing processing is changed to the second region display frame 803 to perform the focusing operation of the SLO image acquiring section. After the focusing operation is completed, subsequently in Step S908, the CPU 301 moves the OCT focus lens 121 along the optical axis based on the position of the SLO focus lens 109 on the optical axis. After the OCT focus lens 121 is stopped on the optical axis, in Step S909, the OCT image acquiring section executes the image acquisition for the OCTA image in an OCTA image acquiring area.

Through the execution of the above-mentioned processing, it is possible to adjust the focusing positions of the SLO focus lens 109 and the OCT focus lens 121 by changing a region to be focused on between the case of acquiring the OCT image and the case of acquiring the OCTA image. Therefore, it is possible to acquire the retina tomographic image at the focusing position of the OCT focus lens 121 appropriate for each image acquisition purpose.

In the third embodiment, the acquisition of a plurality of OCT images is exemplified by the case of acquiring the OCT image and the OCTA image, but an example to which the present invention is applied is not limited to the third embodiment. The case in which two image acquiring modes including an OCT image acquiring mode and an OCTA image acquiring mode are selectively used as an image acquiring mode is described above as an example, but another image acquiring mode can also be added as the image acquiring mode to be selected. That is, the SLO image in the region corresponding to the image acquiring area for OCT in each image acquiring mode may be used to control the focusing position of the SLO focus lens 109 and to also adjust the focusing position of the OCT focus lens 121 in conjunction therewith. That is, the present invention can be suitably be applied to even a case of selecting an image acquiring mode from a larger number of image acquiring modes including additional image acquiring mode as long as the additional image acquiring mode allows the three-dimensional tomographic information to be acquired from a partial region within the original SLO retinal surface image 502.

As described in the third embodiment, the focusing area is displayed as a plurality of predetermined areas within the SLO retinal surface image, winch is displayed through superimposition, respectively as a plurality of display forms exemplified as the region display frame 503 and the second region display frame 803. That is, those display forms are displayed on the displayed front image (on the SLO retinal surface image) in a shape in accordance with the image acquiring mode to be used when the tomographic image is acquired. In that case, in the same manner as in the OCT image acquisition and the OCTA image acquisition, the setting unit (CPU 301) selects one display form from the plurality of display forms in accordance with the image acquiring mode to be used when the tomographic image is acquired by the OCT image acquiring section. The setting unit further sets a region display frame based on an instruction input through use of the cursor 504. With this configuration, an optimal focusing area for SLO is set in accordance with the image acquiring mode, and the focusing condition for the OCT image acquiring section corresponding thereto is obtained.

### Fourth Embodiment

In the above-mentioned first to third embodiments, the SLO image acquiring section is used for fundus observation. However, the SLO image acquiring section has a complicated apparatus configuration because, for example, a laser scanning mechanism is required. Therefore, in order to simplify the apparatus, it is also possible to employ a configuration using a fundus camera for fundus observation. In a fourth embodiment of the present invention, a case of using the fundus camera for fundus observation is described.

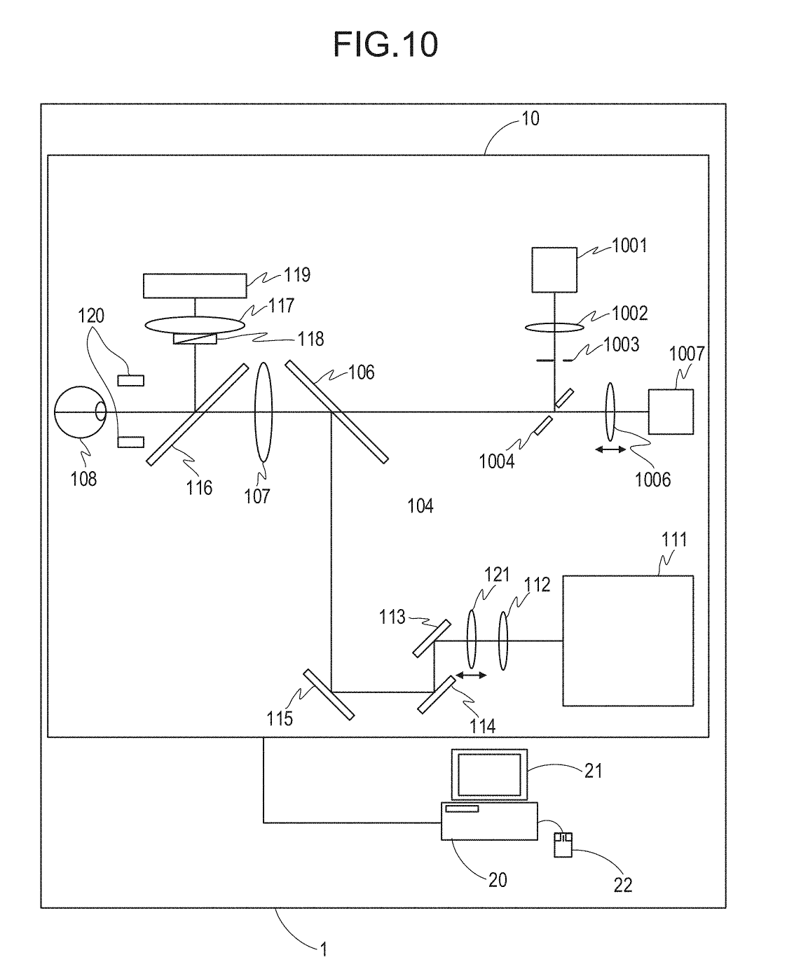

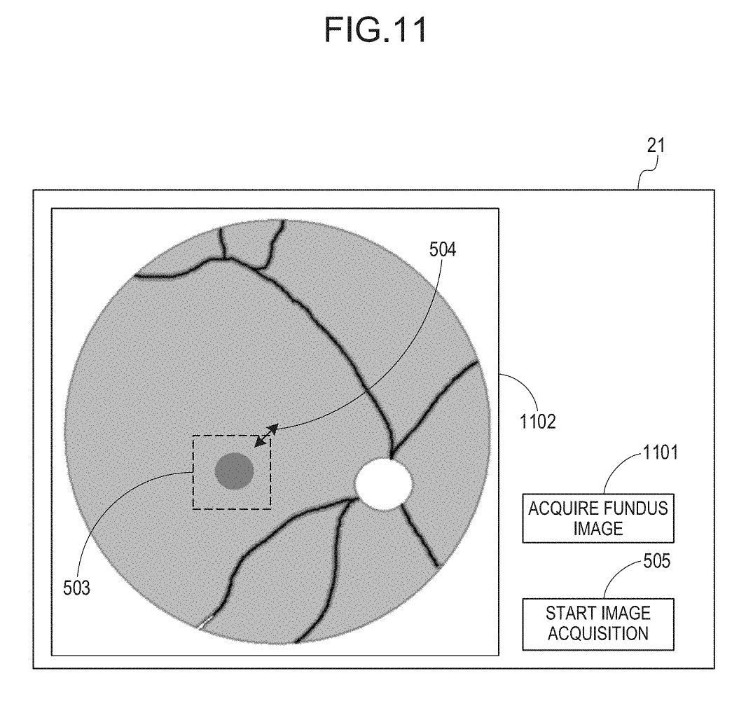

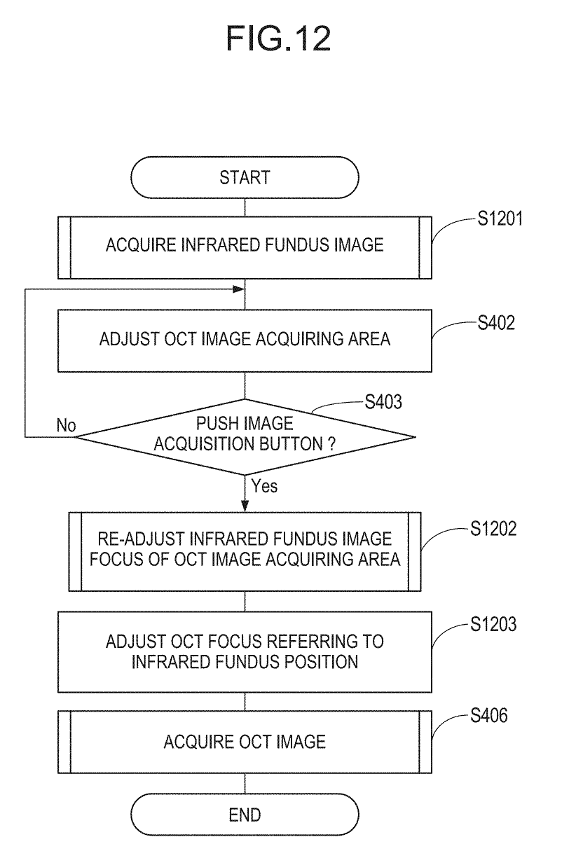

An OCT system in a fourth embodiment of the present invention described below is different from the OCT system in the first embodiment, which is illustrated in FIG. 1, in that a fundus camera is employed in place of the SLO image acquiring section of the OCT apparatus. Now, the fourth embodiment is described with reference to FIG. 10 to FIG. 12. FIG. 10 is a block diagram for illustrating the entire OCT system in the fourth embodiment. FIG. 11 is a diagram for illustrating an example of a GUI screen to be displayed on the monitor by the control apparatus when the user designates the OCT image acquiring area on the fundus of the eye to be inspected at the time of the OCT image acquisition. FIG. 12 is a flow chart for illustrating control processing to be executed by the control apparatus. In the fourth embodiment, the same components and processing steps as the respective components and processing steps described in the above-mentioned first embodiment are denoted by the same reference symbols (and step numbers), and detailed descriptions thereof are omitted below. In the following, points different from the first embodiment are mainly described.

The SLO image acquiring section in the first embodiment is replaced by a fundus image acquiring section illustrated in FIG. 10. In addition, it is not required to scan a beam, and hence a scanner is not arranged. The fundus image acquiring section includes a light source 1001 for fundus observation, a lens 1002, a ring diaphragm 1003, a holed mirror 1004, a focus lens 1006, and an infrared sensor 1007 for observation. The light source 1001 for fundus observation emits infrared light as light for fundus observation. The infrared light emitted from the light source 1001 for fundus observation passes through the lens 1002 and the ring diaphragm 1003 having a ring-like opening, and is reflected by the holed mirror 1004 to reach the second beam splitter 106. The infrared light that has passed through the second beam splitter 106 passes through the eyepiece lens (objective lens) 107, and is applied to the fundus of the eye 108 to be inspected. The infrared light is reflected or scattered by the fundus of the eye 108 to be inspected, follows back the same optical path to be transmitted through the holed mirror 1004, and passes through the focus lens 1006 to reach the infrared sensor 1007 for observation. Through use of the infrared sensor 1007 for observation, it is possible to obtain the two-dimensional image of the fundus of the eye 108 to be inspected. It is also possible to obtain the focused state in the fundus image acquiring section by controlling the position of the focus lens 1006 on the optical axis so as to maximize the contrast of the two-dimensional image.

An example of an operation screen to be displayed on the monitor 21 in the fourth embodiment is illustrated in FIG. 11. In FIG. 11, a fundus image acquiring button 1101 for acquiring the two-dimensional image of the fundus is arranged in place of the SLO front image acquiring button 501 in the first embodiment. When the fundus image acquiring button 1101 is pushed, the infrared light is emitted from the light source 1001 for fundus observation, while the fundus observation is started by the infrared sensor 1007 for observation.

With reference to the flow chart of FIG. 12, acquisition processing for the three-dimensional tomographic information within the designated image acquiring area in the fourth embodiment is described. First, when the fundus image acquiring button 1101 on the operation screen of the monitor 21 is pushed, the image acquisition processing for the OCT image is started, and in Step S1201, the CPU 301 executes the acquisition processing for the two-dimensional image of the fundus. In the acquisition processing for the two-dimensional image, operations for the acquisition of the retinal surface image (two-dimensional image), the confirmation of the focused state using the contrast or the like, and the adjustment of the position of the focus lens 1006 on the optical axis are repeatedly performed. When a retinal surface image 1102 is obtained under the focused state, the retinal surface image 1102 is displayed on the operation screen, and the flow advances to Step S402.

In Step S402, the adjustment or setting of the OCT image acquiring area is performed through use of the cursor 504 as described above. When the operation for moving the cursor 504 or other such operation is temporality stopped, the flow advances to Step S403, and the CPU 301 determines whether or not the image acquisition start button 505 has been pushed. When the CPU 301 determines that the image acquisition start button 505 has not been pushed, the flow returns to Step S402 to repeatedly perform the subsequent processing steps. When the CPU 301 determines in Step S403 that the image acquisition start button 505 has been pushed, the flow advances to Step S1202.

In Step S1202, the CPU 301 performs the focusing processing on the focus lens 1006 with respect to the OCT image acquiring area adjusted or set on the retinal surface image 1102 by the region display frame 503. In the fourth embodiment, the focus lens 1006 is moved along the optical axis so as to maximize the contrast of the two-dimensional image in the OCT image acquiring area. The movement of the focus lens 1006, the acquisition of the two-dimensional image, and the contrast evaluation are repeatedly performed until the focused state is obtained. When the focused state is obtained, the flow advances to Step S1203.

In Step S1203, the CPU 301 moves the OCT focus lens 121 along the optical axis based on the position of the focus lens 1006 on the optical axis in the fundus image acquiring section (fundus camera). The driving amount and position on the optical axis of the locus lens 1006 and the driving amount and position on the optical axis of the OCT focus lens 121 are associated with each other to be stored in advance in the hard disk drive 305 in the same manner as in the case of the first embodiment. The CPU 301 uses the table information to drive the OCT focus lens 121 via the OCT focus driver 319. After the driving of the OCT focus lens 121 is completed, the flow advances to Step S406.

through the execution of the above-mentioned processing, even in a case of using an infrared fundus camera simpler than an optical system for SLO observation, it is possible to acquire the focusing information on the OCT focus lens 121 from the fundus camera in accordance with the OCT image acquiring area. In addition, when the position of the OCT focus lens 121 on the optical axis is adjusted through use of the focusing information, the retina tomographic image can be acquired at the position of the OCT focus lens 121 appropriate for the OCT image acquiring area.

### Fifth Embodiment

In the above-mentioned first to fourth embodiments, the region for adjusting the focusing condition with respect to the retinal surface is set to be the same as the OCT image acquiring area or the OCTA image acquiring area. In those embodiments, when a region including a macula is set as the OCT image acquiring area, the focusing is normally achieved on a region other than the macula occupying a large part of the OCT image acquiring area. However, depending on an inspection condition, there is a demand for focusing on the macula and obtaining the tomographic image of surroundings thereof. In a fifth embodiment of the present invention, in order to handle such a demand for acquiring a clear tomographic image of such a region of interest, a small region for focusing adjustment is set at a specific position within the OCT image acquiring area or set in an area different from the OCT image acquiring area.

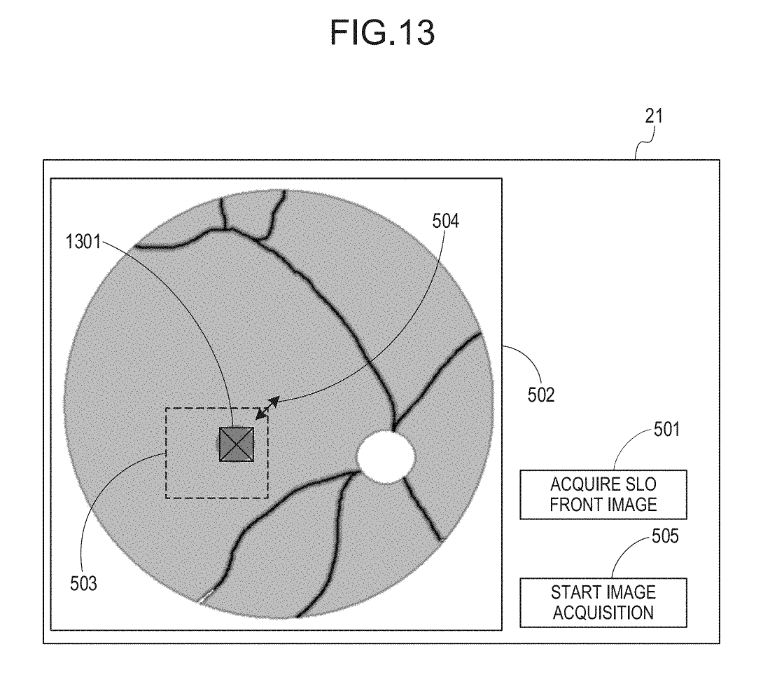

FIG. 13 is a diagram for illustrating an example of an operation screen to be displayed on the monitor 21 at the time of acquiring an OCT image. The operation screen is different from the operation screen described in the first embodiment with reference to FIG. 5 in that a focus adjustment area 1301 is superimposed on the SLO retinal surface image 502 to be displayed on the operation screen in addition to the region display frame 503 and the cursor 504. In the fifth embodiment, it is possible to set the OCT image acquiring area or OCTA image acquiring area and the focus adjustment area 1301 independently of each other by, for example, moving the focus adjustment area 1301 through use of the cursor 504. That is, the cursor 504 is used to point at the focus adjustment area 1301 to independently move the focus adjustment area 1301, to thereby cause the SLO image acquiring section to obtain the focusing portion with respect to the focus adjustment area 1301.

The fifth embodiment is described on the assumption that one focus adjustment area is set as a predetermined region for acquiring the focusing condition, but a plurality of focus adjustment areas may be set. In this case, focusing conditions for the SLO image acquiring section and the like may be obtained with respect to each focus adjustment area (with respect to each of a plurality of predetermined areas), and the focusing positions obtained from the respective focus adjustment areas may be averaged. With this configuration, when the OCT image or the like is generated, it is possible to acquire the tomographic image having the focus achieved on the region of interest. In addition, by enabling the size or the like of the above-mentioned focus adjustment area 1301 to be changed, it is possible to improve the definition or the like of the tomographic image in the region of interest or in a required area at the time of the OCT image acquisition or the OCTA image acquisition.

### Sixth Embodiment

The above-mentioned embodiments are described by taking the case in which the OCT image acquiring area or the OCTA image acquiring area has a rectangular shape. However, there is a case in which the scanning area used by the measuring light cannot be represented by a rectangle in the acquisition of the tomographic image through OCT, for example, in so-called radial scan for performing the image acquisition by scanning the measuring light radially about the macula. In such a case, the region over which the measuring light is scanned may be allowed to have the size or shape set to include the region display frame 503. Specifically, in the case of the radial scan having scan lines arranged radially about a specific point, it is possible to set the smallest OCT image acquiring area that includes all those scan lines by setting the region display frame 503 to have a circular shape.

That is, the display form of the focusing area exemplified by the region display frame 503 can be displayed by being superimposed on the SLO retinal surface image in a shape of, for example, a circle, an ellipse, or a rectangle, in accordance with the image acquiring mode including a scan format for acquiring the tomographic image through OCT. When the shape of the region display frame 503 can be thus deformed, it is possible to eliminate the influence of the focused state in the region that is not involved in the image acquisition, which can improve precision in focusing with respect to the OCT image acquiring area.

As described above, according to the present invention, the focused state is adjusted in accordance with an image acquiring area, which is used for actually acquiring a tomographic image, and is narrower than an observable area. This facilitates the movement of the focus lens up to the position along the optical axis appropriate for the image acquiring area in the OCT image acquisition, and it is possible to acquire the retina tomographic image of the eye to be inspected under an appropriately focused state.

According to the present invention, a tomographic image can be acquired by the OCT apparatus under an appropriate focusing condition.

In the embodiments described above, it is assumed that a planar image of a fundus is first acquired and displayed, and then the user determines the OCT image acquiring area in the displayed fundus planar image. However, when the OCT image acquiring area is determined on the fundus in advance at the time of, for example, the execution of followup, the focusing operation performed first when the fundus planar image is obtained may be omitted. In this case, for example, in the step of the image acquisition processing, which is indicated as Step S401, its processing may be reduced, and it suffices that the fundus image is merely displayed.

Further, in the above-mentioned embodiments, the configuration in which the control apparatus, which is exemplified by a personal computer integrally formed of the control apparatus 20, the monitor 21, the input apparatus 22, the storage apparatus, and the like, and the OCT apparatus are connected to each other in a wired manner is taken as an example. However, the configuration of the OCT system is not limited thereto, and the configuration may be appropriately changed. For example, the control apparatus and the OCT apparatus may be integrally formed, or the confirmation on the control apparatus side may be separated as necessary, or may be partially integrated with the OCT apparatus as the need arises. For example, the optical tomographic image acquiring apparatus 10 and the control apparatus 20, which are described above, can be integrated with each other as the OCT system (tomographic image acquiring system). Further, the connection among the individual components is not limited to the wired connection, and the components may be connected to one another in a wireless manner. Further, the OCT apparatus and the control apparatus may be connected to each other via a server through a LAN, a WAN, the Internet, of the like.

Further, in the above-mentioned embodiments, the configuration of a Michelson interferometer is used as the configuration of the interference optical system of the OCT apparatus, but the configuration of the interference optical system is not limited thereto. For example, the interference optical system of the OCT apparatus may include the configuration of a Mach-Zehnder interferometer. In addition, the configuration of the optical systems arranged inside the OCT apparatus is not limited to the configuration exemplified in each embodiment, and a part of the configuration included in those optical systems may be provided separately from the configuration included in the optical system inside the OCT apparatus.

Further, in the above-mentioned embodiments, a fiber optical system using an optical coupler is used in the OCT image acquiring section as a unit configured to split light emitted from a light source. However, a spatial optical system using a collimator and a beam splitter may be used in place of the above-mentioned optical system. In addition, the beam splitters are used as optical members configured to branch light off to the individual image acquiring sections, but the optical members are not limited thereto. For example, a mirror formed of a holed mirror or a hollow prism onto which a mirror has been deposited by vapor deposition may be used to split light on a wavelength-to-wavelength basis.

Further, in the above-mentioned embodiments, the spectral-domain OCT (SD-OCT) apparatus, which uses the SLD as the light source, is described as the OCT image acquiring section, but the configuration of the OCT image acquiring section in the present invention is not limited thereto. The present invention is also applicable to any other type of OCT apparatus, for example, a swept-source OCT (SS-OCT) apparatus, which uses a wavelength-swept light source capable of sweeping a wavelength of emitted light.

Further, the above-mentioned embodiments are described by employing the eye to be inspected as the object to be inspected. However, the object to be inspected is not limited to the eye to be inspected, and may be, for example, a skin or an organ. In this case, the present invention can be applied to not only an ophthalmic apparatus but also an endoscope or other such medical equipment.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-108992, filed Jun. 1, 2017, which is hereby incorporated by reference herein in its entirety.

What is claimed is:

1. A control apparatus comprising:
a setting unit configured to set a focusing area on an object to be inspected;
a condition acquiring unit configured to acquire a first focusing condition for a front image acquiring unit with respect to the focusing area, the focusing area being set in a partial region of a front image of the object to be inspected acquired by the front image acquiring unit, and being narrower than an image acquiring area of the front image; and
a control unit configured to cause a tomographic image acquiring unit to acquire a tomographic image under a second focusing condition in accordance with the acquired first focusing condition.

2. A control apparatus according to claim 1, further comprising a display control unit configured to cause a display unit to display the front image,
wherein the display control unit is configured to cause the display unit to superimpose the focusing area set by the setting unit on the displayed front image to display the front image with the focusing area.

3. A control apparatus according to claim 2,
wherein the focusing area is displayed as a display form for indicating a predetermined region of the displayed front image, and
wherein the setting unit is allowed to change the focusing area by performing at least one of movement or deformation of the display form.

4. A control apparatus according to claim 3, wherein the display form is displayed on the displayed front image in a shape in accordance with an image acquiring mode to be used when the tomographic image is acquired.

5. A control apparatus according to claim 2,
wherein the focusing area is displayed as a plurality of display forms for each indicating each of a plurality of predetermined areas in the displayed front image, and
wherein the setting unit is configured to select one display form from among the plurality of display forms in accordance with an image acquiring mode to be used when the tomographic image is acquired by the tomographic image acquiring unit.

6. A control apparatus according to claim 1, further comprising a determination unit configured to determine, when the tomographic image is acquired plural times, whether a distance between focusing areas set with respect to tomographic images exceeds a threshold value,
wherein the condition acquiring unit is configured to avoid changing the second focusing condition when the determination unit determines that the distance between the focusing areas is equal to or smaller than the threshold value.

7. A control apparatus according to claim 1, wherein the set focusing area has the same area as an image acquiring area for acquiring the tomographic image.

8. A control apparatus according to claim 2,
wherein the focusing area is displayed as a plurality of display forms for each indicating each of a plurality of predetermined areas in the displayed front image,
wherein the first focusing condition includes a plurality of first focusing conditions each obtained for each of the plurality of predetermined areas, and
wherein the tomographic image acquiring unit is configured to acquire the tomographic image under the second focusing condition in accordance with a focusing condition obtained by averaging the plurality of first focusing conditions.

9. A control apparatus according to claim 1, wherein the condition acquiring unit is configured to acquire the first focusing condition for the front image acquiring unit with respect to the focusing area set in the front image acquired with focus being achieved on the image acquiring area.

10. A control apparatus according to claim 1, wherein the front image acquiring unit is configured to acquire an image of a surface of the object to be inspected through use of at least one of reflected light or scattered light of laser light scanned on the object to be inspected.

11. A control apparatus according to claim 1, wherein the tomographic image acquiring unit is configured to acquire the tomographic image through use of interference light obtained by multiplexing return light of scanned measuring light from the object to be inspected and reference light corresponding to the return light with each other.

12. A tomographic image acquiring system comprising:
a tomographic image acquiring unit; and
a control apparatus,
the control apparatus including:
a setting unit configured to set a focusing area on an object to be inspected;
a condition acquiring unit configured to acquire a first focusing condition for a front image acquiring unit with respect to the focusing area, the focusing area being set in a partial region of a front image of the object to be inspected acquired by the front image acquiring unit, and being narrower than an image acquiring area of the front image; and
a control unit configured to cause the tomographic image acquiring unit to acquire a tomographic image under a second focusing condition in accordance with the acquired first focusing condition.

13. A control method comprising:
setting a focusing area on an object to be inspected;
acquiring a first focusing condition for a front image acquiring unit with respect to the focusing area, the focusing area being set in a partial region of a front image of the object to be inspected acquired by the front image acquiring unit, and being narrower than an image acquiring area of the front image; and
causing a tomographic image acquiring unit to acquire a tomographic image under a second focusing condition in accordance with the acquired first focusing condition.

14. A non-transitory tangible medium having recorded thereon a program for causing a computer to perform steps of the control method of:
setting a focusing area on an object to be inspected;
acquiring a first focusing condition for a front image acquiring unit with respect to the focusing area, the focusing area being set in a partial region of a front image of the object to be inspected acquired by the front image acquiring unit, and being narrower than an image acquiring area of the front image; and
causing a tomographic image acquiring unit to acquire a tomographic image under a second focusing condition in accordance with the acquired first focusing condition.
